(19) United States
(12) Patent Application Publication
(10) Pub. No.: US 2021/0063413 A1
(43) Pub. Date: Mar. 4, 2021

(54) DENDRITIC CELL POTENCY ASSAY

(71) Applicant: MEDIGENE IMMUNOTHERAPIES GMBH, Planegg-Martinsried (DE)

(72) Inventors: Dolores SCHENDEL, München (DE); Judith ECKL, Penzberg (DE); Christiane GEIGER, Deggendorf (DE); Isabel RÖMER, München (DE)

(73) Assignee: MEDIGENE IMMUNOTHERAPIES, Planegg-Martinsried (DE)

(21) Appl. No.: 16/643,982

(22) PCT Filed: Sep. 5, 2017
(86) PCT No.: PCT/EP2017/072254

§ 371 Date: Mar. 3, 2020

(51) Int. Cl. G01N 33/68; C12N 5/0784

(52) U.S. Cl. CPC G01N 33/6869; C12N 5/0639; C12N 2501/999; C12N 2501/52; G01N 2333/5434; G01N 2333/70575; G01N 2333/5428

(57) ABSTRACT

The present invention relates to a method for determining the potency of DCs, comprising the steps: stimulating dendritic cells by incubation with soluble CD40L and TLR7/8 agonist, measuring the secretion of the marker proteins IL-10 and IL-12 from the stimulated dendritic cells. Thereby it can be determined whether the dendritic cell have a high capability to activate T-cells and Natural Killer (NK) cells. The invention also encompasses a method for stimulating dendritic cells comprising the step of stimulating the dendritic cells with soluble CD40L and TLR7/8 agonist.

### FIELD OF THE INVENTION

The present invention relates to a method for determining the potency of DCs, comprising the steps: stimulating dendritic cells by incubation with soluble CD40L and TLR7/8 agonist, measuring the secretion of the marker proteins IL-10 and IL-12 from the stimulated dendritic cells. Thereby it can be determined whether the dendritic cells have a high capability to activate T-cells and Natural Killer (NK) cells. The invention also encompasses a method for stimulating dendritic cells comprising the step of stimulating the dendritic cells with soluble CD40L and TLR7/8 agonist.

### BACKGROUND OF THE INVENTION

Antigen-loaded dendritic cells are capable of priming naive T cells and natural killer (NK) cells and therefore can be used as vaccines in anti-tumor immunotherapy or treatment of chronic viral infections. These tumor-vaccines induce an increased T cell response against the disease-associated antigens by stimulating cytotoxic T lymphocytes (Subklewe et al.).

Clinical trials have shown that DC-based immunotherapy is safe and that administration of dendritic cells that have been modified in vitro led to enhancement of specific immunity.

In the past immune monitoring has concentrated only on the aspects of responding T cells whereas the DC side of immune response has mostly been overlooked. This may be due in part to difficulties in determining relevant DC functions for analysis. To activate T cells optimally in a cancer immunotherapy setting, three signals must be delivered by the DCs. First, the right antigen must be presented in adequate amounts by MHC complexes to trigger TCRs (Signal 1); second, activating costimulatory molecules like CD80/CD86 must dominate over negative regulatory molecules on the DC surface (Signal 2) to provide positive co-stimulation to the T cells and third, the bioactive form of the cytokine IL-12 should be secreted in the absence of IL-10 to polarize T-cells in a Th1/Tc1 direction (Signal 3). Signal 1 and Signal 2 can be monitored using flow cytometry. Signal 3 can be measured by mimicking the DC-T cell interaction via CD40/CD40L binding.

However, the quality of the dendritic cells generated for administration can vary. This variation of the dendritic cells influences their capability to activate T cells and natural killer cells (NK cells).

The quality of the dendritic cells depends on its origin, differentiation and maturation status. Even when the same maturation protocol and normal healthy donors are used, the T cell activation capability varies significantly between individuals.
Therefore, it is necessary monitor the suitability of dendritic cells to be used as highly potent vaccines.

Since the methods for testing the dendritic cell potency will be necessary to provide a reliable vaccine therapy, it is desired that the tests are robust, cost-effective and can be carried out according to good manufacturing standards (GMP).

The standard assay for potency of DC vaccine is the upregulation of CD80 on the surface of the dendritic cells. This assay is accepted by the regulatory authorities but does not reflect the true functionality of the dendritic cells. The CD80 test does not discriminate dendritic cells with high IL-12 and low IL-10 secretion, which show a superior T cell an NK activating capability, from DC cells which secrete low IL-12 and high IL-10.

Dendritic cells have to be activated, in order to induce T cell activation. In a standard assay measuring dendritic cell potency dendritic cells are incubated with L929 mouse fibroblasts that express CD40L which are irradiated. Via the interaction of the CD40L expressed on the mouse fibroblasts and CD40 at the dendritic cell, the dendritic cells are activated.

This approach is disadvantageous, since the co-culture of the radiated mouse fibroblasts and the dendritic cells is laborious, time-consuming, costly, difficult to standardize and is difficult to apply to GMP standards. Moreover, a radiation unit is necessary.

Standard single cell assay for cytokines is the intracellular staining and detection via flow cytometry. It allows the detection of IL-12 and Il-10 in one assay. The problem of this approach is that the intracellular staining does not show actual secretion but it solely measures intracellular cytokine levels induced upon stimulation. Hence, detection of intercellular cytokines does not mean that these cytokines are secreted by the cells. If a cytokine is already stored before stimulation in the cells deduction of actual secretion is not possible. Only if the measured levels significantly increase it can be indirectly deduced that there is an upregulation of the cytokine due to the stimulation. The factual levels of secreted cytokines are not detected. If the cytokine level remains unchanged it could be that there is no increase in cytokines or that there is an increase but it is as fast as the degradation of the intracellular cytokines so no increase is detectable. Mature DCs can normally store IL-10 over a prolonged period of time and if IL-10 is secreted it is not reflected in the intracellular staining data, such as flow cytometry data.

Hence, there is a need for methods for testing the dendritic cell potency in a robust, cost-effective and simple way that can be carried out according to good manufacturing standards (GMP) and measures directly the secretion of IL-12 and IL-10.

### OBJECTIVES AND SUMMARY OF THE INVENTION

Hence, a first aspect of the invention refers to a method for determining the potency of DCs, comprising the following steps: (a) stimulating dendritic cells by incubation with soluble CD40L and TLR7/8 agonist, (b) measuring the secretion of the marker proteins IL-10 and IL-12 from the dendritic cells of (a).

This method allows a precise prediction of the capability of the dendritic cells to elicit T-cell and NK cell immune response, since it measures the factual secretion level of the marker cytokines IL-12 and IL-10 which allows a precise prediction of the potency of the dendritic cells. Moreover, the method is robust, simple and cost-effective.

“Potency of dendritic cells” refers to the capability of the dendritic cells to activate T cells and/or NK cells. Dendritic cells with a high potency in the context of the invention thus means that the dendritic cells are able to activate T cells and/or NK cells. In particular, high potent dendritic cells in the context of the invention are able to polarize T cells into a Th1/Tc1 phenotype, which is characterized by a secretion of IFNγ by the T cells and no or reduced expression of IL-4 of the same cells, and/or to activate NK cells to express high levels of CD69 and to secrete IFNγ.

In a preferred embodiment, the measurement is carried out on a single cell level. The assays used in the prior art only measure the cell markers in the bulk culture and therefore cannot provide the correct analysis of heterogeneous populations. From the results, it is not clear if only a small number of cells produce high amounts of cytokines or if a high number of cells produce low amounts of a given analyte. Additional it is not possible to detect if cells produce both analytes at the same time.

In order to specifically determine the potency status of a heterogeneous dendritic cell population, the single cell measurement is advantageous.

In preferred embodiments, the TLR7/8 agonist is 4-amino-2-ethoxymethyl-α,α-dimethyl-1H-imidazol[4,5-c]quinoline -1-ethanol (R848).

The incubation of dendritic cells with a combination of soluble CD40L and R848 is particularly advantageous since, it mimics the stimulation with the CD40L-transfected L929 cells for dendritic cells that differed in their maturation conditions, e.g. that were incubated with different maturation cocktails. Hence the of CD40L and R848 represent a universal dendritic cell stimulating composition.

The method may further comprise the step (c) classification of the dendritic cell potency based on the secretion profile of IL-12 and IL-10. Thereby, a dendritic cell showing a ratio of IL-12 to IL-10 secretion of more than 1 is classified as dendritic cell with a high capability to activate T-cells and Natural Killer (NK) cells.

The dendritic cell with a high capability to activate T-cells and NK cells may have a phenotype of high CD80 expression levels, high CD86 expression levels, low CD14 expression levels and low B7H1 expression levels.

Dendritic cells with a high capability to activate T-cells may polarize T cells into a Th1/Tc1 phenotype. The Th1/Tc1 phenotype is characterized by a secretion of IFNγ by the T cells and no or reduced expression of IL-4.

Dendritic cells with a high capability to activate NK cells may activate NK cells to express high levels of CD69 and to secrete IFNγ.

In particular, step (b) of the method comprises the following steps:
i) incubating the dendritic cells with a primary binding protein for IL-10 and a primary binding protein specific for IL-12,
ii) detecting the binding of the maker protein to the primary binding protein by a secondary binding protein.

Typically, the stimulation only occurs by binding of CD40L and TLR7/8 agonist to the dendritic cells.

In the method of the invention, CD40L is not presented to the dendritic cells by a cell line. Since the dendritic cells are activated by incubation with the soluble form of CD40L it is not necessary co-culture the dendritic cells with as different cell line, such as L929 mouse fibroblasts expressing CD40L. Therefore, cell culture which is laborious, time consuming and difficult to standardize can be avoided. Moreover, this means that no radiation step has to be applied to assure that the L929 mouse cells do not continue to proliferate, which in effect would lead to L929 cells overgrowing the human dendritic cells, rendering the assay impossible to be performed. Therefore, for the method of the invention a radiation unit, which is not present in every hospital, is not necessary.

In some embodiments, the primary binding proteins specific for IL-10 and IL-12 are immobilized on a carrier. Thereby, the carrier may be uniformly coated with the primary binding proteins for IL-10 and IL-12. This allows measuring the secretion of IL-10 and IL-12 on a single cell level. Typically, the carrier is a multi-well plate. Usually, the primary binding protein is an antibody. The secondary binding protein may also be an antibody.

The secondary binding protein may be labelled with a detection label. Typically, the secondary binding protein is labelled with a fluorescent functional group or label.

Usually, the dendritic cells used in the method of the invention are matured dendritic cells. Matured dendritic cells, when stimulated with the method according to the invention express the required ratio of IL-12 to IL-10.

The maturation of the dendritic cells may for example occur by incubation with a maturation cocktail.

Preferably dendritic cells are used in the method of the invention are matured by a maturation cocktail comprising IL1β, TNFα, INFγ, TLR7/8 agonist and Prostaglandin E2.

More preferably dendritic cells are used in the method of the invention are maturated by the following steps:
i) provision of monocytes; ii) incubation of the monocytes of step i) with IL-4 and GM-CSF;
iii) incubation of the monocytes of step ii) with IL-4 and GM-CSF in combination with a maturation cocktail comprising IL1β, TNFα, INFγ, TLR7/8 agonist and Prostaglandin E2.

The incubation of step ii) may last for at least 2 days. The incubation of step iii) may last for at least 12 hours, preferably 24 hours.

Another aspect of the invention refers to a method for stimulating dendritic cells comprising the following steps: a) providing dendritic cells, and

b) stimulating the dendritic cells with soluble CD40L and TLR7/8 agonist.

Accordingly, the invention refers to the use of soluble CD40L and TLR7/8 agonist for stimulating dendritic cells.

Another aspect of the invention refers to a kit comprising:
TLR7/8 agonist,
soluble CD40L,
a primary binding protein specific for IL-10,
a primary binding protein specific for IL-12,
a secondary binding protein specific for IL-10, and
a secondary binding protein specific for IL-12.

For example, the kit may comprise the following components:
(i) a composition comprising TLR7/8 agonist and CD40L,
(ii) a composition comprising a primary binding protein specific for IL-10 and a primary binding protein specific for IL-12, and
(iii) a composition comprising a secondary binding protein specific for IL-10 and a secondary binding protein specific for IL-12.

The primary binding protein may preferably be an antibody and/or wherein the secondary binding protein is an antibody. Typically, wherein the TLR7/8 agonist is R848.

### FIGURE LEGENDS

FIG. 1: DC stimulation

FIG. 1A: Three signals are necessary for optimal DC stimulation DCs are powerful activators of T cells and influence the character of the effector T cell through the interaction between the DCs and the T cell. For optimal activation three signals have to be delivered by the DCs

Signal 1: Antigen presentation via MHC/peptide and the corresponding TCR

Signal 2: activating co-stimulatory signal CD80/CD86 engaging CD28

Signal 3: Secretion of activating cytokines e.g. IL-12 and absence of inhibiting cytokines e.g. IL-10; Adapted from De Koker, 2011.

FIG. 1B:

In a conventional Signal 3 Assay DCs are co-cultured with a murine fibroblast cell line L929 expressing human CD40L for 24 h. Cytokines IL-12p70 and IL-10 secreted into the culture supernatant are determined by a commercial sandwich ELISA (e.g. R&Dsystems).

The assay is disadvantageous since it requires a radiation unit, evolves a laborious cell culture, rendering the assay difficult to utilize in a GMP facility. Further, the assay is specific on a single cell level.

FIGS. 2 to 5: Comparison of conventional versus CD40L stimulation of dendritic cells in an IL-10/IL-12 ELISA

FIG. 2 shows IL-10/IL-12 secretion in DC maturated with the MDG cocktail and stimulated with CD40L in different combinations with R848, IFN-γ and LPS compared to the conventional Signal 3 Assay. (IL-10/IL-12 ELISA)

FIG. 3 shows the same experiments with Jonuleit maturated DCs. In both assays the CD40L stimulation shows comparable results with the conventional Signal 2 Assay in regards to IL-12 secretion but not for IL-10 if CD40L stimulation is used alone. (IL-10/IL-12 ELISA)

FIGS. 4 and 5 show that R848 is necessary as a costimulatory reagent for optimal IL-10 secretion especially in immature DCs (iDCs) and Jonuleit maturated DCs. FIG. 4 shows IL-10/IL-12 ELISA data. FIG. 5 shows data of the carried out by the method of the invention (ELISPOT Sig.3 CD40L)

FIGS. 6 to 8: Double color IL-10/IL-12 ELISPOT but not flow cytometry shows potency of dendritic cells comparable to ELISA

FIG. 6 shows the ELISPOT Data of two different Donors for IL-12 and IL-10. Stimulation was performed with 20000 cells and 1 μl CD40L/100 μl ([v/v]) medium together with R848. Donor 1 is a high IL-10 producer whereas Donor 2 mainly produces IL-12.

FIG. 7 shows the same donors in a conventional Signal 3 Assay. Both Assays show highly comparable results. In contrast to the dc IL-10/IL-12 ELISPOT, the flowcytometric analysis of intracellular IL-12 and IL-10 could only show comparable results to the IL-12 secretion data but not for IL-10. This is probably due to intracellular IL-10 storage.

FIG. 8 depicts a comparison of the IL-12 and IL-10 secretion after a conventional signal 3 assay detected by ELISA or corresponding intracellular flow cytometric (FACS) staining of IL-12 and IL-10 of cell of the same donor. The intracellular staining results as detected by FACS are not comparable to the secretion as detected by ELISA, in particular with regard to IL-10. This can be explained by the fact that FACS measures total intracellularly stored IL-10 whereas ELISA detects secreted IL-10.

FIG. 9 Single cell IL-10/IL-12 assay (ELISPOT) In the upper row the first picture shows the spot count of IL12 producing mature Dendritic cells (maturated with our own cocktail) the second picture the spot number of IL-10 producing cells in the same well and the third picture the number of spots of double positive cells. In the second row below, the respective data for immature dendritic cells is shown. (Human IL-10/IL-12 Double-Color ELISPOT Kit (CTL); cell number: 20,000 cells/well, 1.1 μl/110 μl CD40L+R848) The results clearly show that immature DCs (iDCs) are the main source of IL-10 in healthy donors.

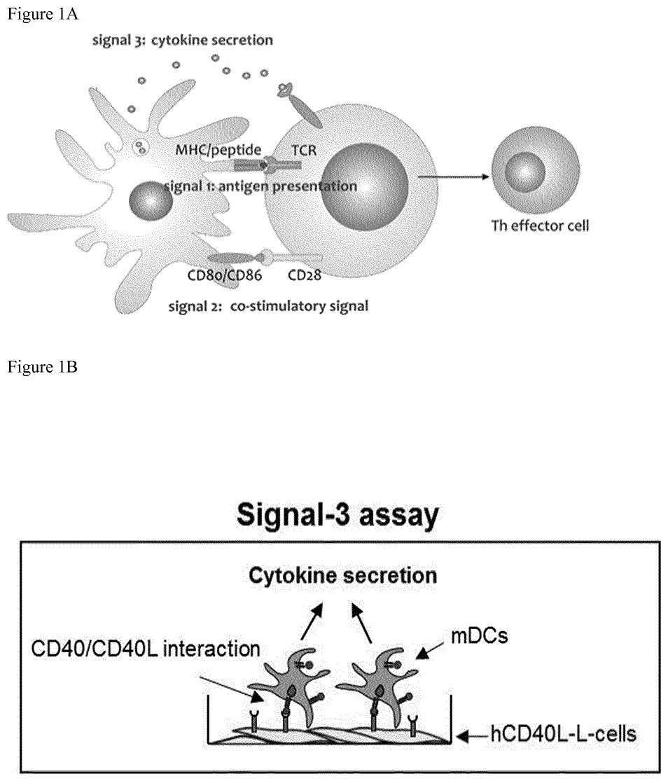

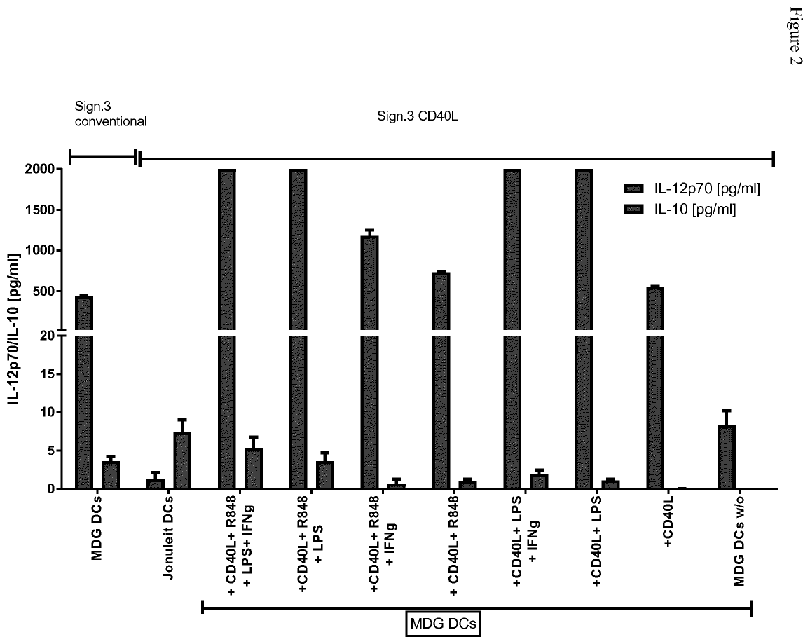

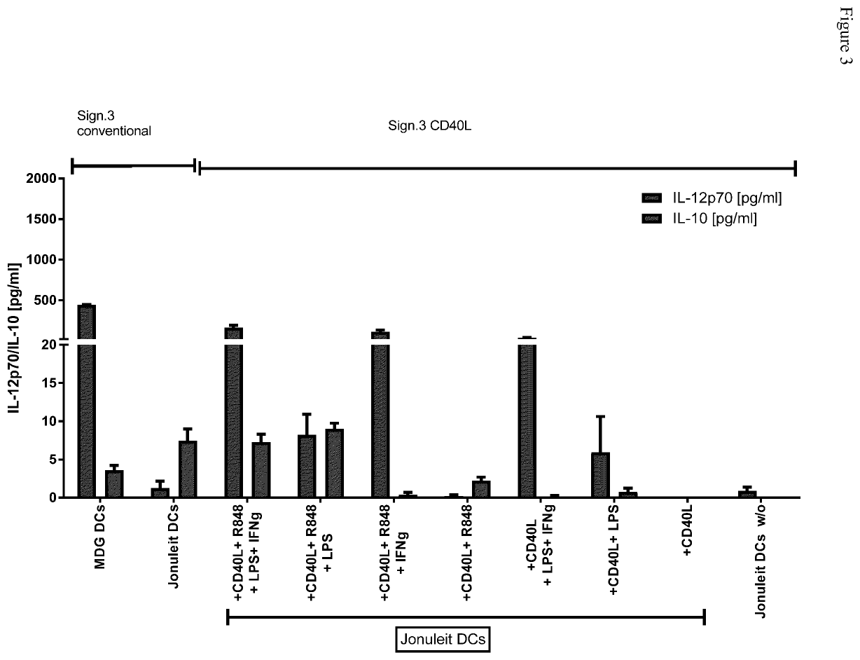

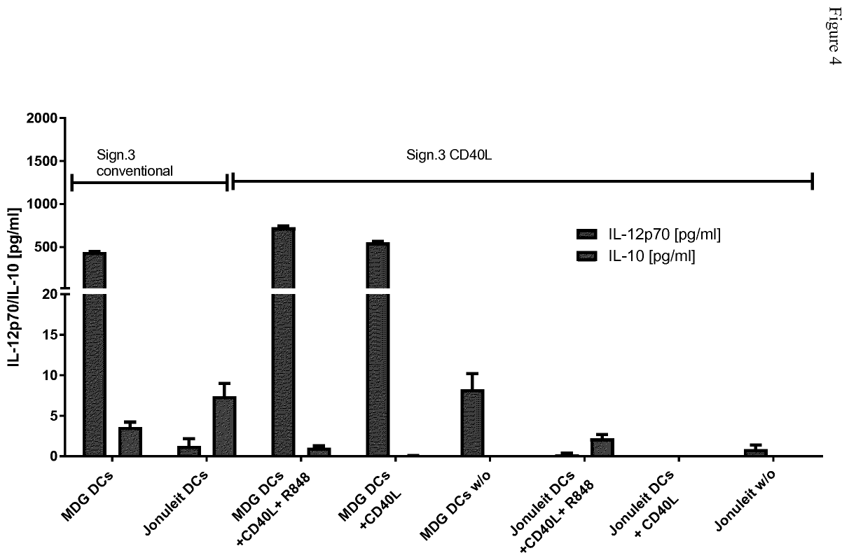

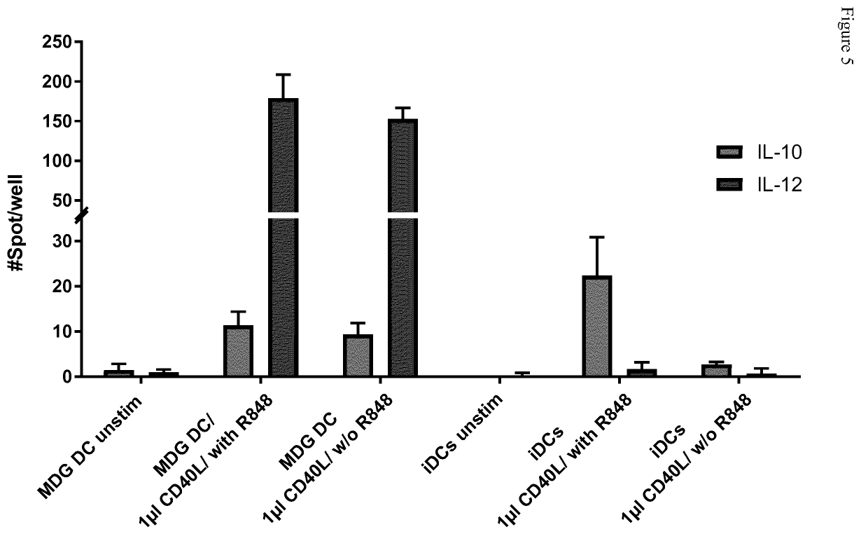

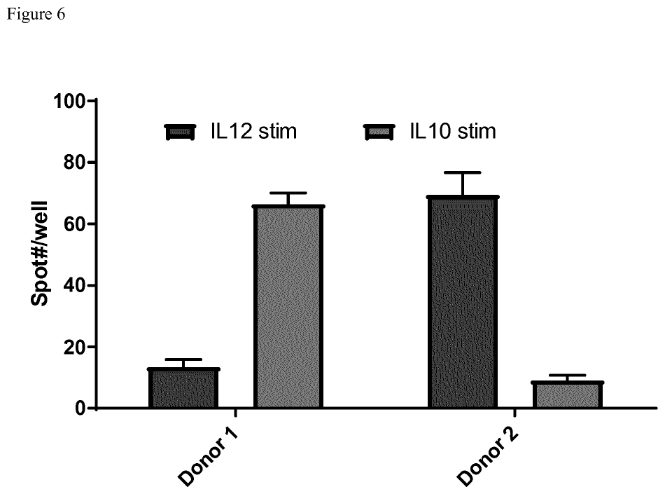

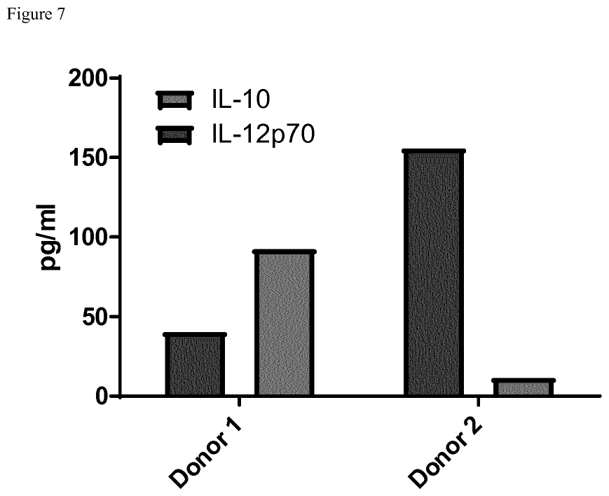

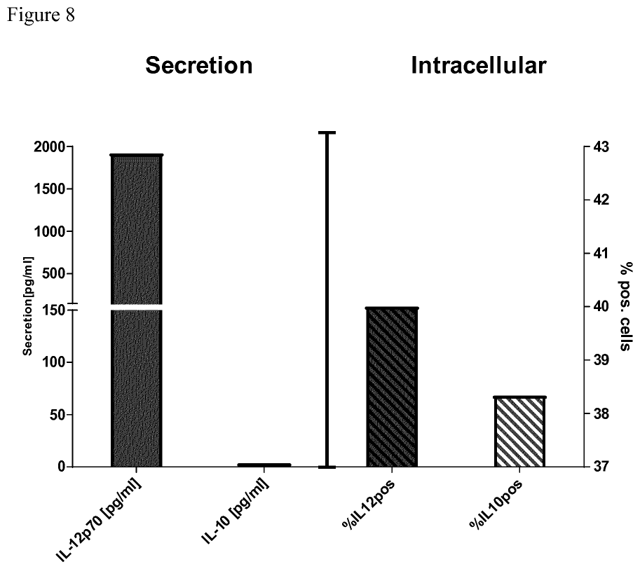

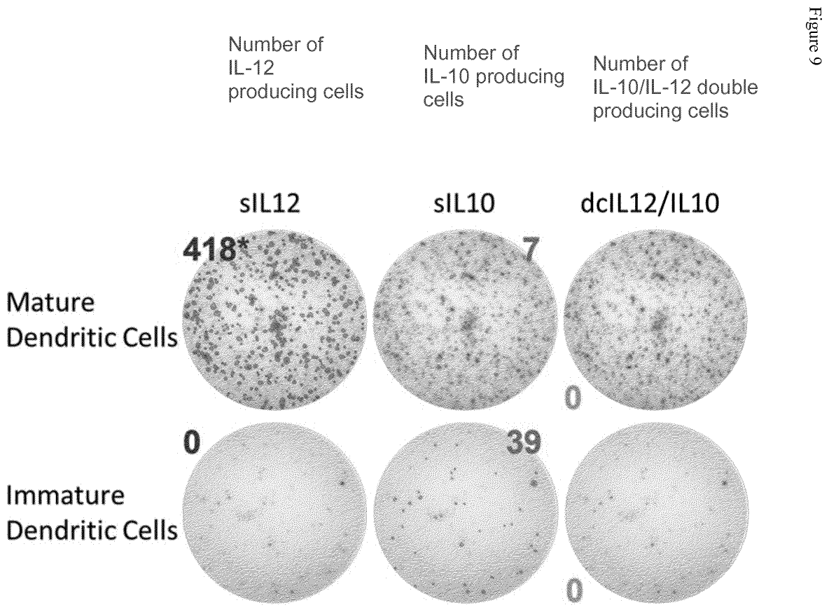

### DETAILED DESCRIPTION OF THE INVENTION

Before the invention is described in detail with respect to some of its preferred embodiments, the following general definitions are provided.

The present invention as illustratively described in the following may suitably be practiced in the absence of any element or elements, limitation or limitations, not specifically disclosed herein.

The present invention will be described with respect to particular embodiments and with reference to certain figures but the invention is not limited thereto but only by the claims.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group which preferably consists only of these embodiments.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

Where an indefinite or definite article is used when referring to a singular noun, e.g. “a”, “an” or “the”, this includes a plural of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±10%, and preferably of ±5%.

Technical terms are used by their common sense. If a specific meaning is conveyed to certain terms, definitions of terms will be given in the following in the context of which the terms are used.

Generally, the invention refers to a method for determining the potency of DCs, comprising the following steps: (a) stimulating dendritic cells by incubation with soluble CD40L and optionally TLR7/8 agonist, (b) measuring the secretion of the marker proteins IL-10 and IL-12 from the dendritic cells of (a).

In particular, the invention refers to a method for determining the potency of DCs, comprising the following steps: (a) stimulating dendritic cells by incubation with soluble CD40L and TLR7/8 agonist, (b) measuring the secretion of the marker proteins IL-10 and IL-12 from the dendritic cells of (a).

Dendritic cells express CD40 at the cell surface. CD40-CD-40L interaction stimulate dendritic cells. “Soluble CD40L” refers to a modified version of the membrane associate CD40L (also named CD154 or CD40 Ligand). The term “soluble CD40L” includes truncated versions of CD40L which do not contain its transmembrane domain. The term “soluble CD40L” also includes CD40L oligomers, in particular modified CD40L oligomers, such as trimeric CD40 ligand molecules that are artificially linked via the collagen domain of Adiponectin/ACRP30/AdipoQ, such as MEGACD40L (Enzo Life Science GmbH, Lörrach, Germany). Soluble CD40L may be capable of stimulating the natural membrane-assisted aggregation of CD40L. CD40L may be used in a concentration of 10 ng/ml to 100 μg/ml, preferably 1 ng/ml to 10 μg/ml, such as 1 μg/ml. The incubation with CD40L may last at least 12 h, preferably 12 h to 48 h, more preferably about 24 h. TLR7/8 is also named Toll-like receptor 7/8. Preferably the TLR7/8 agonist is TLR7/8 agonist is 4-amino-2-ethoxymethyl-α,α-dimethyl-1H-imidazol[4,5-c]quinoline-1-ethanol (R848), which is described in WO 00/47719 and is obtainable from Invivogen, San Diego, USA. The TLR7/8 agonist, preferably R848, might be used at a concentration between 0.2 and 5 μg/ml, preferably 0.5 μg/ml to 2 μg/ml, more preferably 1 μg/ml. The incubation with TLR7/8 agonist, in particular R848, may last at least 12 h, preferably 12 h to 48 h, more preferably about 24 h.

The TLR7/8 agonist and CD40L may be added to dendritic cells in combination or sequentially.

The incubation with the combination of CD40L and TLR7/8 agonist is particularly advantageous since, it mimics the stimulation with the CD40L-transfected L929 cells for dendritic cells that differed in their maturation conditions, e.g. that were incubated with different maturation cocktails. Hence CD40L and TLR7/8 agonist represent a universal dendritic cell stimulating composition.

Importantly, the present invention measures the secretion of the marker proteins IL-10 and IL-12 from the dendritic cells. This means that the IL-12 and IL-10 molecules that are secreted into the medium are measured and not the intracellular IL-12 and IL-10 content. As already described above do the intracellular content the interleukins not necessarily correspond to the secreted levels. This particularly applies to IL-10.

Typically, the dendritic cells may be plated on carrier plates that are coated with the IL-10 primary binding protein and the IL-12 primary binding protein. The stimulating solution containing CD40L and optionally TLR7/8 agonist may be added after plating or before plating the dendritic cells. After incubation time, the secondary binding protein is added. The incubation time may be 6 to 48 h, preferably 12 to 36 h, most preferably 24 h.

Thus, measuring the secretion of the marker proteins IL-10 and IL-12 refers to the detection of the marker proteins, by incubation with specific binding proteins to IL-10 and IL-12 that mediate the detection of the marker proteins. A binding protein may be thus modified by a label, such as a fluorescent label or by an enzyme that promotes a reaction that allows the detection of the marker protein using a specific substrate, e.g. horse reddish peroxidase for IL-10 and alkaline phosphatase for IL-12).

In a preferred embodiment, the measurement is carried out on a single cell level. Any method that is capable of measuring interleukin secretions on a single cell level could be applied. This method thus does not measure the interleukin secretion in the bulk culture, i.e. the sum of the interleukins in a cell population, but measures the interleukin secretion individually per cell. As already pointed out above, this is advantageous, since the interleukin status of heterogeneous cell populations, containing cells showing different interleukin secretions, can be analysed in more detail.

Thus, a preferred embodiment refers to a method for determining the potency of DCs, comprising the following steps: (a) stimulating dendritic cells by incubation with soluble CD40L and R848, (b) measuring the secretion of the marker proteins IL-10 and IL-12 from the dendritic cells of (a),

wherein the assay is carried out on a single cell level.

The method may further comprise the step (c) classification of the dendritic cell potency based on the secretion profile of IL-12 and IL-10. In other words, the method comprises step (c) classification whether the dendritic cell has a high capability to activate T-cells and or NK cells. Thereby, a dendritic cell showing a ratio of IL-12 to IL-10 secretion of more than 1 is classified as dendritic cell with a high capability to activate T-cells and NK cells. Preferably the dendritic cell showing a ratio of IL-12 to IL-10 secretion of more than 5, such as more than 10, more than 20, more than 30, more than 40, more than 50, more than 60, more than 70, more than 80 is classified as dendritic cell with a high capability to activate T-cells and/or NK cells. The ratio of IL-12 to IL-10 is typically less than 10,000, such as less than 5,000, less than 1,000, or less than 500.

Vice versa, a dendritic cell showing a ratio of IL-10 to L-12 secretion of more than 1 is classified as dendritic cell inhibiting immune response.

A preferred embodiment refers to a method for determining the potency of DCs, comprising the following steps: (a) stimulating dendritic cells by incubation with soluble CD40L and R848, (b) measuring the secretion of the marker proteins IL-10 and IL-12 from the dendritic cells of (a),

(c) classification whether the dendritic cell has a high capability to activate T-cells and/or NK cells; wherein a dendritic cell showing a ratio of IL-12 to IL-10 secretion of more than 1 is classified as dendritic cell with a high capability to activate T-cells and/or NK cells; wherein the assay is carried out on a single cell level.

The dendritic cell with a high capability to activate T-cells and NK cells may have a phenotype of high CD80 expression levels, high CD86 expression levels, low CD14 expression levels and low B7H1 expression levels. (Lichtenegger et al.; Kenneth Murphy & Casey Weaver: Janeway's Immunobiology).

Dendritic cells with a high capability to activate T-cells may polarize T cells into a Th1/Tc1 phenotype. The Th1/Tc1 phenotype is characterized by a secretion of IFNγ by the T cells and no or reduced expression of IL-4. Dendritic cells with a high capability to activate NK cells may activate NK cells to express high levels of CD69 and to secrete IFNγ. (Kenneth Murphy & Casey Weaver: Janeway's Immunobiology).

Typically, the stimulation only occurs by binding of soluble CD40L and TLR7/8 agonist to the dendritic cells. This means that no other stimuli, e.g. incubation with other molecules is necessary for stimulating dendritic cells. In particular, it is not necessary that the transmembrane protein CD40L is presented to the dendritic cells in its transmembrane form anchored in the membrane of a cell, such as radiated L929 mouse fibroblasts expressing CD40L. Therefore, it is not necessary that the method includes a radiation step.

In particular, step (b) of the method comprises the following steps:
i) incubating the dendritic cells with a primary binding protein for IL-10 and a primary binding protein specific for IL-12,
ii) detecting the binding of the maker protein to the primary binding protein by a secondary binding protein.

The detection of step ii) may be by binding of the secondary binding protein to the complex of the marker protein and the primary binding protein. Alternatively, the secondary binding protein might solely bind to the primary binding protein (without interaction with the primary binding protein). In this case, due to washing steps before step ii), marker proteins that are not bound to the primary binding protein are removed.

In some embodiments, the primary binding proteins specific for IL-10 and IL-12 are immobilized on a carrier. Thereby, the carrier may be uniformly coated with the primary binding proteins for IL-10 and IL-12. This allows measuring the secretion of IL-10 and IL-12 on a single cell level. Typically, the carrier is a multi-well plate. Usually, the primary binding protein is an antibody. The secondary binding protein may also be an antibody.

The secondary binding protein may be labelled with a detection label. Typically, the secondary binding protein is fluorescently labelled or modified by an enzyme that promotes a reaction that allows the detection of the marker protein.

Usually, the dendritic cells that are used in the method of the invention are matured dendritic cells. Matured dendritic cells, when stimulated with CD40L (and the TLR7/8 agonist) express the required ratio of IL-12 to IL-10. The maturation of the dendritic cells may for example occur by incubation with a maturation cocktail.

Typically, mature dendritic cells, may be generated by a method comprising the following steps: i) provision of monocytes; ii) incubation of the monocytes of step i) with IL-4 and GM-CSF; iii) incubation of the monocytes of step ii) with IL-4 and GM-CSF in combination with a maturation cocktail.

The maturation cocktail may comprise at least one of the components selected from the group consisting of IL-β, TNF-α, IFN-γ, TLR7/8 agonist, PGE2 and TLR3 agonist or a combination thereof. The TLR7/8 agonist may be R848 or CL075. The TLR3 agonist may be poly(I:C). In a specific embodiment, the maturation cocktail may comprise a combination of IL1β, TNFα, INFγ, TLR7/8 agonist and Prostaglandin E2. The TLR7/8 agonist may be R848 or CL075. Preferably the TLR7/8 agonist is R848.

The maturation cocktail may comprise 1-50 ng/ml TNFα, 1-50 ng/ml IL-1β, 500-10,000 U/ml INFγ, 0.2-5 μg/ml TLR7/8 agonist and 50-5,000 ng/ml Prostaglandin E2 PG and optionally 10-50 ng/ml TLR3 agonist; More preferably the cocktail may comprise 10 ng/ml TNF-α, 10 ng/ml IL-1β, 5,000 U/ml IFNγ, 1 ug/ml R848 and 250 ng/ml prostaglandin E2.

The incubation of step ii) may last for 3 days. The incubation of step iii) may last for at least 24 hours.

Alternatively, also the Jonuleit cocktail could be used. The Jonuleit cocktail is described in Jonuleit et al. and comprises TNFα, IL-1β, IL-6 and Prostaglandin E2. In particular, the Jonuleit cocktail comprises 10 ng/ml TNF-alpha, 10 ng/ml IL-1β, 15 ng/ml IL-6 and 1000 ng/ml Prostaglandin E2.

The dendritic cell may be donor derived antigen presenting cells, e.g. isolated monocytes which are maturated to dendritic cells. Maturated dendritic cells can be optimally stimulated by the method of the invention.

Typically, the dendritic cells are autologous cells, i.e. cells obtained from a patient which are treated according to teaching of the invention and then re-administered to the same patient. For example, monocytes are isolated from a patient, matured to dendritic cells and treated as described herein to express the desired antigen and then administered to the same patient.

The skilled person is aware of maturation protocols for dendritic cells. The maturation of dendritic cells use for immunotherapy of acute myloid leukemia is disclosed in Subklewe et al.;

Bürdek et al. describes a three-day maturation protocol for dendritic cells.

Monocytes may be for example peripheral blood mononuclear cells.

Another aspect of the invention refers to a method for stimulating dendritic cells comprising the following steps: a) providing dendritic cells, and

b) stimulating the dendritic cells with soluble CD40L and TLR7/8 agonist.

Another aspect of the invention refers to a kit comprising:
TLR7/8 agonist,
soluble CD40L,
a primary binding protein specific for IL-10,
a primary binding protein specific for IL-12,
a secondary binding protein specific for IL-10, and
a secondary binding protein specific for IL-12.

The secondary binding protein specific for the marker protein (either IL-12 or IL-10) may bind the complex of the marker protein and the primary binding protein or may solely bind to the primary binding protein (without interaction with the primary binding protein).

For example, the kit may comprise the following components:
(i) a composition comprising TLR7/8 agonist and CD40L,
(ii) a composition comprising a primary binding protein specific for IL-10 and a primary binding protein specific for IL-12, and
(iii) a composition comprising a secondary binding protein specific for IL-10 and a secondary binding protein specific for IL-12.

The primary binding protein may preferably be an antibody and/or the secondary binding protein is an antibody. Typically, the TLR7/8 agonist is R848.

The term “binding protein” includes not only antibodies and binding fragments thereof but also includes other molecules, such as non-antibody protein scaffold proteins and aptameres.

Antibodies can be differentiated into five main classes on the basis of their heavy chain, the IgM (μ), IgD (δ), IgG (γ), IgA (α) and IgE (ε) antibodies, IgG antibodies making up the largest proportion. Immunoglobulins can moreover be differentiated into the isotypes κ and λ on the basis of their light chains.

### EXPERIMENTS

### Dendritic Cells Maturated with MDG or Jonuleit Cocktail and Stimulated with CD40L Alone and in Combination with R848/IFNγ and LPS

Monocytes are isolated with adherent isolation followed by dendritic cell generation for 3 days using IL-4 and GMSCF. Afterwards dendritic cells were maturated with MDG cocktail and IL-4 and GMCSF for 24 h. In case of Jonuleit cocktails cells were incubated for 6 days using IL-4 and GMSCF. Then cells where incubated with the Jonuleit cocktail (and IL-4 and GMCSF) for 24 h. For all assays, frozen dendritic cells were used thawed the day before the assay and incubated in DC medium until assay start. Stimulation is carried out for 24 h in a 96 well plate. Therefore, 11,000 dendritic cells were seeded per well. 1.1 μl CD40L in 110 μl DC Medium (VLE RPMI 1640 with 1.5% human serum) 1 μg/ml LPS and R848 were added.

Elisa assay was performed according to the protocols of the human IL-10, IL12p70 respectively, DuoSet ELISA-Kit (R&D Systems, Inc, Minneapolis, USA).

### IL-10/IL-12 Elispot Assay

The IL-10/IL-12 Elispot assay was carried out according to the protocol of the Human IL-10/IL-12 Double-Color ELISPOT kit of (C.T.L. Cellular technology Limited, Cleaveland, USA)

### Jonuleit Cocktail

10 ng/ml TNFα, 10 ng/ml IL-1β, 15 ng/ml IL-6 and 1,000 ng/ml Prostaglandin E2 (=PGE2)

### MDG Cocktail

10 ng/ml TNF-α, 10 ng/ml IL-1β, 5,000 U/ml IFNγ, 1 μg/ml R848 and 250 ng/ml prostaglandin E2.

### REFERENCES

Bürdek “Three-day dendritic cells for vaccine developement: Antigen uptake; processing and presentation ” Journal of Translational Medicine (2010) 8:90

De Koker, et al “Designing polymeric particles for antigen delivery” Chem Soc Rev 2011 40(1):320-39; 2011)

Jonuleit et al. ,,Pro-inflammatory cytokines and prostaglandins induce maturation of potent immunostimulatory dendritic cells under fetal calf serum-free conditions.” Eur J Immunol., (1997), vol. 27(12): 3135-42

Lichtenegger “CD86 and IL-12p70 are key players for T Helper 1 polarization and Natural Killer Cell Activation by Tell-Like Receptor-Induced Dendritic Cells” PLOS ONE, vol. 7(9) (e44266)

Murphy K. & Weaver C.: Janeway's Immunobiology 9th Edition, Garland Science

Subklewe et al. “New generation dendritic cell vaccine for immunotherapy of acute myeloid leukemia.” Cancer Immunol Immunother (2014) 63: 1093-1103

1. Method for determining the potency of DCs, comprising the following steps:
(a) stimulating dendritic cells by incubation with soluble CD40L and TLR7/8 agonist,
(b) measuring the secretion of the marker proteins IL-10 and IL-12 from the dendritic cells of (a).

2. Method according to any one of the preceding claims, wherein the measurement is carried out on a single cell level.

3. Method according to any one of the preceding claims, wherein the TLR7/8 agonist is 4-amino-2-ethoxymethyl-α,α-dimethyl-1H-imidazol[4,5-c]quinoline-1-ethanol (R848).

4. Method according to any one of the preceding claims, wherein the method further comprises the step of
(c) classification of the dendritic cell potency based on the secretion profile of IL-12 and IL-10.

5. Method according to any one of the preceding claims, wherein a dendritic cell showing a ratio of IL-12 to IL-10 secretion of more than 1 is classified as dendritic cell with a high capability to activate T-cells and Natural Killer (NK) cells.

6. Method according to claim 5, wherein the dendritic cell with a high capability to activate T-cells and NK cells have a phenotype of high CD80 expression levels, high CD86 expression levels, low CD14 expression levels and low B7H1 expression levels.

7. Method according to claim 5 or 6, wherein the dendritic cell with a high capability to activate T-cells polarizes T cells into a Th1/Tc1 phenotype.

8. Method according to claim 7, wherein the Th1/Tc1 phenotype is characterized by a secretion of IFNγ by the T cells and no or reduced expression of IL-4.

9. Method according to claims 5 to 8, wherein the dendritic cell with a high capability to activate NK cells activates NK cells to express high levels of CD69 and to secrete IFNγ.

10. Method according to any one of the preceding claims, wherein step (b) comprises the following steps:
i) incubating the dendritic cells with a primary binding protein for IL-10 and a primary binding protein specific for IL-12,
ii) detecting the binding of the maker protein to the primary binding protein by a secondary binding protein.

11. Method according to any one of the preceding claims, wherein the stimulation only occurs by binding of soluble CD40L and TLR7/8 agonist to the dendritic cells.

12. Method according to any one of the preceding claims, wherein CD40L is not presented to the dendritic cells by a cell line.

13. Method according to any one of the preceding claims, wherein the dendritic cells are not co-cultured with a different cell line.

14. Method according to any one of the preceding claims, wherein no radiation step is applied.

15. The method according to claims 10 to 14, wherein the primary binding proteins specific for IL-10 and IL-12 are immobilized on a carrier.

16. The method according to claim 15, wherein the carrier is uniformly coated with the primary binding proteins for IL-10 and IL-12.

17. The method according to claim 15 or 16, wherein the carrier is a multi-well plate.

18. The method according to any one of claims 10 to 17, wherein the primary binding protein is an antibody.

19. The method according to any one of claims 10 to 18, wherein the secondary binding protein is an antibody.

20. The method according to claims 10 to 19, wherein the secondary binding protein is fluorescently labelled.

21. Method according to any one of the preceding claims, wherein the dendritic cells are matured dendritic cells.

22. Method according to claim 21, wherein the maturation of the dendritic cells occurs by incubation with a maturation cocktail.

23. Method for stimulating dendritic cells comprising the following steps:
a) providing dendritic cells, and
b) stimulating the dendritic cells with soluble CD40L and TLR7/8 agonist.

24. Use of soluble CD40L and TLR7/8 agonist for stimulating dendritic cells.

25. Kit comprising:
TLR7/8 agonist,
soluble CD40L,
a primary binding protein specific for IL-10,
a primary binding protein specific for IL-12,
a secondary binding protein specific for IL-10, and
a secondary binding protein specific for IL-12.

26. Kit according to claim 25 comprising:
(i) a composition comprising TLR7/8 agonist and CD40L,
(ii) a composition comprising a primary binding protein specific for IL-10 and a primary binding protein specific for IL-12, and
(iii) a composition comprising a secondary binding protein specific for IL-10 and a secondary binding protein specific for IL-12.

27. Kit according to claim 25 or 26, wherein the primary binding protein is an antibody and/or wherein the secondary binding protein is an antibody.

28. Method according to claim 23, use according to claim 24 kit according to claims 25 to 27, wherein the TLR7/8 agonist is R848.
